(19) United States
(12) Patent Application Publication
(10) Pub. No.: US 2014/0155993 A1
(43) Pub. Date: Jun. 5, 2014

(54) DEVICE FOR MITIGATING OR PREVENTING PARAVALVULAR LEAKS

(71) Applicant: THE CLEVELAND CLINIC FOUNDATION, Cleveland, OH (US)

(72) Inventor: Samir Kapadia, Chagrin Falls, OH (US)

(73) Assignee: THE CLEVELAND CLINIC FOUNDATION, Cleveland, OH (US)

(21) Appl. No.: 14/096,047

(22) Filed: Dec. 4, 2013

Related U.S. Application Data
(60) Provisional application No. 61/733,008, filed on Dec. 4, 2012.

(51) Int. Cl. A61F 2/24

(52) U.S. Cl. CPC A61F 2/2445; A61F 2/2466
USPC 623/2.11; 623/2.36; 623/2.37

(57) ABSTRACT

One aspect of the present disclosure relates to a device for preventing or mitigating paravalvular leakage associated with a valve replacement procedure. The device can include a first ring, a second ring axially spaced apart from the first ring, at least one pad section extending between the first and second rings, and a plurality of centering wires attached to at least one of the first and second rings.

### RELATED APPLICATION

This application claims priority to U.S. Provisional Patent Application Ser. No. 61/733,008, filed Dec. 4, 2012, the entirety of which is hereby incorporated by reference for all purposes.

### TECHNICAL FIELD

The present disclosure relates generally to cardiovascular devices and, more particularly, to devices and methods for preventing or mitigating paravalvular leakage during a valve replacement procedure.

### BACKGROUND

Prosthetic heart valves have many forms and designs, and have been used for more than 20 years for treatment of heart patients when replacement of a diseased natural valve or malfunctioning prosthesis is the treatment of choice. Although prosthetic valves have been made in numerous configurations and from many different materials, none have been found to answer all of the requirements for durability, biocompatability, non-thrombogenicity and hemodynamic excellence. Basically, there are two types of heart valve prostheses—mechanical valve prostheses and natural tissue valve prostheses. Mechanical valves, although very durable and hemodynamically acceptable (in some forms), have proven to be thrombogenic. Tissue or bioprosthetic valves, on the other hand, are relatively non-thrombogenic but exhibit relatively poor hemodynamics and questionable durability.

The various designs of heart valve prostheses employing synthetic materials all suffer to one degree or another—either from the material employed or the design configuration—from the incidence of thrombus formation. Furthermore, the designs that have found widespread medical acceptance also employ rigid or semi-rigid valve ring structures, which do not enjoy the ability to flex or move with the movement of the annular tissue as the heart expands and contracts, thereby reducing myocardial efficiency and leading to paravalvular leaks and valvular dehiscence. In addition, the ring structures can occupy up to 50-percent of the available annular area for blood flow, thus reducing cardiac efficiency and raising transvalvular pressure gradients.

### SUMMARY

The present disclosure relates generally to cardiovascular devices and, more particularly, to devices and methods for preventing or mitigating paravalvular leakage during a valve replacement procedure.

One aspect of the present disclosure relates to a device for preventing or mitigating paravalvular leakage associated with a valve replacement procedure. The device can comprise a first ring, a second ring axially spaced apart from the first ring, at least one pad section extending between the first and second rings, and a plurality of centering wires attached to at least one of the first and second rings.

Another aspect of the present disclosure relates to a device for preventing or mitigating paravalvular leakage associated with an aortic valve replacement procedure. The device can comprise a first ring, a second ring, three pad sections, and a plurality of centering wires. The second ring can be axially spaced apart from the first ring. The three pad sections can extend between the first and second rings. Each of the pad sections can include a commissure engaging surface. The plurality of centering wires is attached to at least one of the first and second rings.

Another aspect of the present disclosure relates to a method for preventing or mitigating paravalvular leakage during a procedure to replace a diseased heart valve having a valve annulus and at least two commissures. One step of the method can comprise providing a device that includes a first ring, a second ring that is axially spaced apart from the first ring, at least one pad section extending between the first and second rings, and a plurality of centering wires attached to at least one of the first and second rings. The device can be deployed in the valve annulus so that the at least one pad section engages at least one commissure to form a seal therebetween and thereby reduce or prevent paravalvular leakage. Next, a prosthetic valve can be deployed within the device.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a perspective view of a device for preventing or mitigating paravalvular leakage associated with a valve replacement procedure constructed in accordance with one aspect of the present disclosure;

FIGS. 2A-B are schematic illustrations showing a normal aortic valve during diastole (FIG. 2A) and systole (FIG. 2B);

FIGS. 3A-B are schematic illustrations showing a diseased aortic valve during diastole (FIG. 3A) and systole (FIG. 3B);

FIG. 4 is a perspective view showing a pad section of the device in FIG. 1;

FIGS. 5A-C are schematic illustrations showing different ways in which a pad section (FIG. 4) can be attached to a ring of the device in FIG. 1;

FIG. 6 is a perspective view showing the device in FIG. 1 constructed in accordance with another aspect of the present disclosure;

FIG. 7 is a perspective view showing the device in FIG. 1 having a stented prosthetic valve disposed therein and constructed in accordance with another aspect of the present disclosure;

FIG. 8 is a schematic illustration showing the device in FIG. 1 being positioned within a diseased aortic valve (superior perspective); and

FIG. 9 is a schematic illustration showing the device in FIG. 8 implanted within the diseased aortic valve (superior perspective).

### DETAILED DESCRIPTION

In the context of the present disclosure, the term “subject” can refer to any warm or cold-blooded organism including, but not limited to, human beings, pigs, rats, mice, dogs, goats, sheep, horses, monkeys, apes, rabbits, cattle, birds, marine mammals, fish, reptiles, amphibians, etc.

When an element or structure is referred to herein as being “on,” “engaged to,” “connected to,” “attached to”, or “coupled to” another element or structure, it may be directly on, engaged, connected or coupled to the other element or structure, or intervening elements or structures may be present. In contrast, when an element is referred to as being “directly on,” “directly attached to,” “directly connected to,” or “directly coupled to” another element or structure, there may be no intervening elements or structures present. Other words used to describe the relationship between elements should be interpreted in a like fashion (e.g., “between” versus “directly between,” “adjacent” versus “directly adjacent,” etc.).

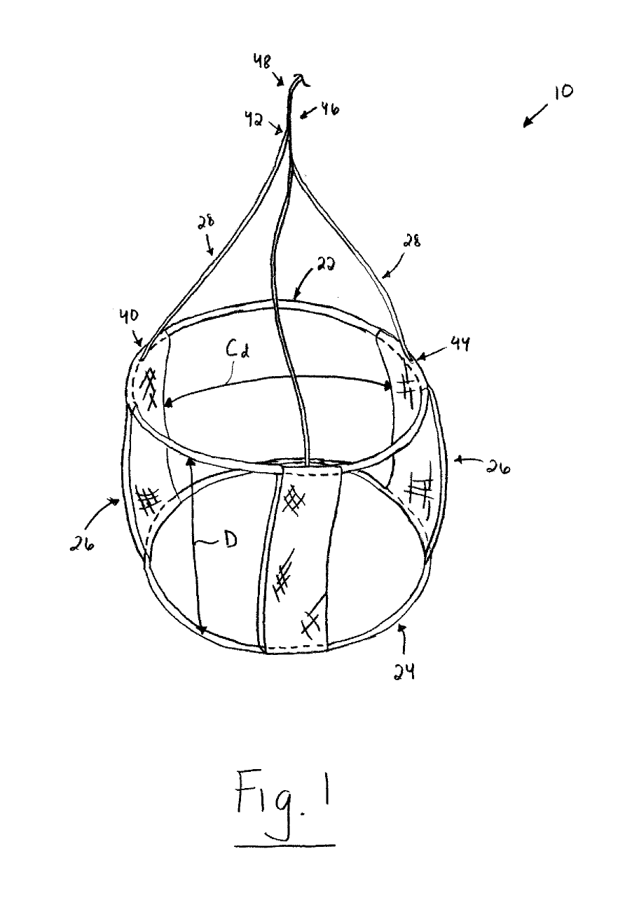

The present disclosure relates generally to cardiovascular devices and, more particularly, to devices and methods for preventing or mitigating paravalvular leakage during a valve replacement procedure. As representative of one aspect of the present disclosure, FIG. 1 illustrates a device 10 for preventing or mitigating paravalvular leakage associated with a valve replacement procedure. During an aortic valve replacement procedure, for example, a prosthetic aortic valve is inserted in place of the diseased aortic valve. Upon insertion of the prosthetic aortic valve, the diseased aortic valve leaflets (e.g., calcified leaflets) are displaced outward towards the aortic valve annulus. Since the calcified valve leaflets are hardened and somewhat rigid, an optimal seal is not formed between the prosthetic aortic valve and the commissures of the diseased aortic valve, thereby leading to paravalvular leakage. Advantageously, the present disclosure provides a device 10 configured to improve seal formation between an implanted prosthetic valve and a native valve annulus by helping to flatten calcified native valve leaflets against the commissures of the diseased valve. Although the present disclosure is described below primarily in terms of preventing or mitigating perivalvular leakage during an aortic valve replacement procedure, it will be appreciated that the device can be used during any valve replacement procedure (e.g., a mitral or tricuspid valve replacement procedure).

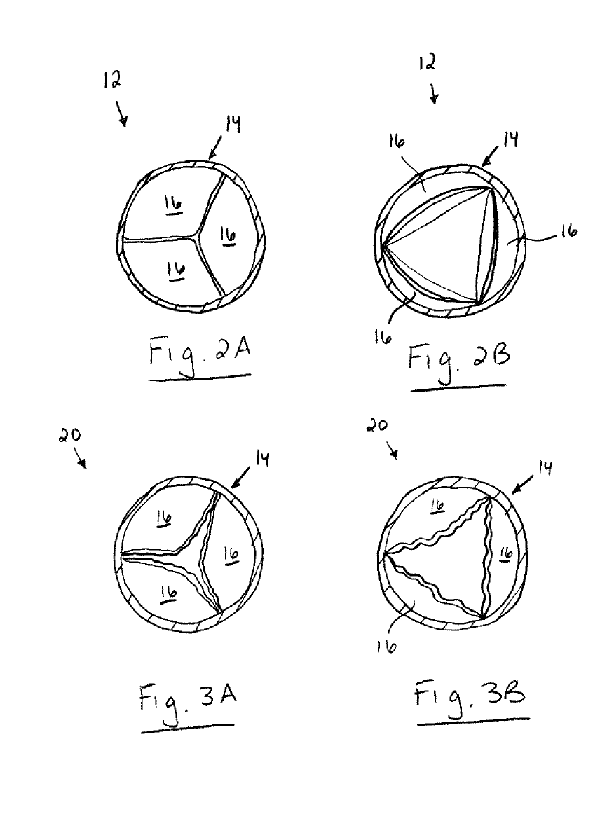

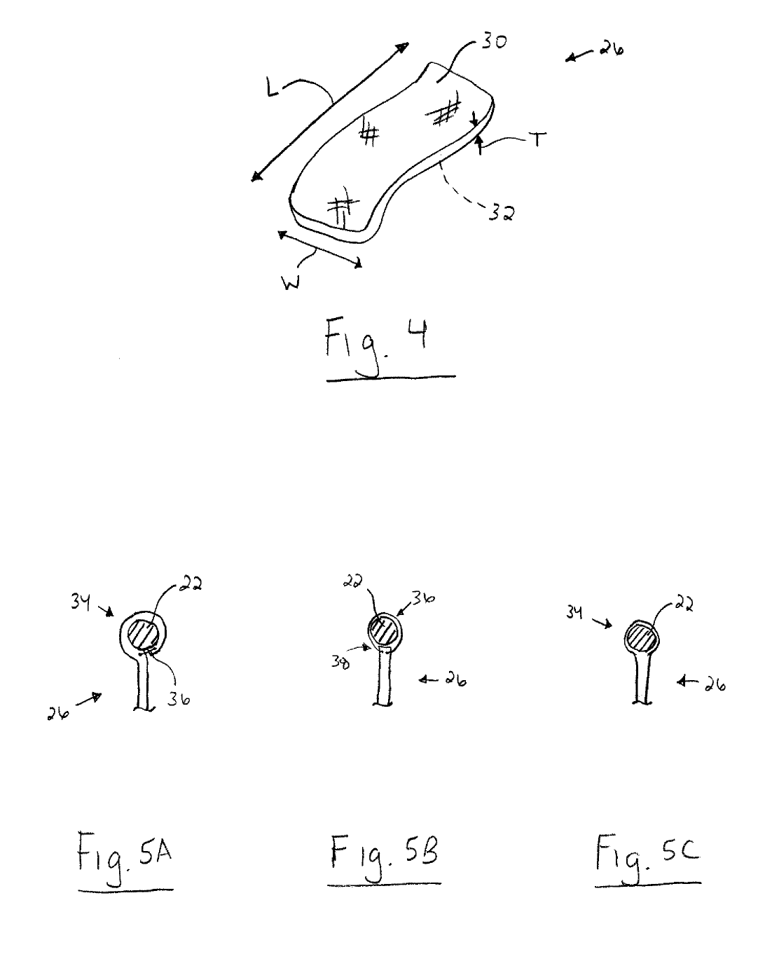

A cardiac valve includes any valve that controls the flow of blood through and from the heart. Examples of cardiac valves include the mitral valve, the tricuspid valve and the aortic valve 12 (FIGS. 2A-B), among others. The aortic valve 12 functions to prevent the regurgitation of blood from the aorta (not shown) into the left ventricle (not shown) during ventricular diastole (FIG. 2A), and to allow the appropriate flow of blood (cardiac output) from the left ventricle into the aorta during ventricular systole (FIG. 2B). The aortic valve 12 has three principle components: the annulus 14; three leaflets 16 (also known as flaps or cusps); and commissures 18. Each of the commissures 18 is the site of a junction between adjacent cusps 16 of the aortic valve 12. The three commissures 18 of the aortic valve 12 lie at the apex of the annulus 14, and are equally spaced around the aortic trunk. The commissures 18 are composed of collagenous fibers oriented in a radial fashion that penetrate into the aortic intima and are anchored in the media of the aorta. The commissure 18 between the left and posterior cusps is located at the right posterior aspect of the aortic root, whereas the commissure between the right and noncoronary cusp is located at the right anterior aspect of the aortic root. In a normal aortic valve 14, the leaflets 16 open and close to control the flow of blood into the aorta from the left ventricle of the heart as it beats. In a diseased aortic valve 20 (FIGS. 3A-B), however, the aortic valve is unable to completely close when the pressure in the left ventricle falls below the pressure in the aorta. This causes blood leakage from the aorta into the left ventricle. In such instances, replacement of the diseased aortic valve 20 with a prosthetic valve is often necessitated; albeit with risk of certain complications, such as paravalvular leakage.

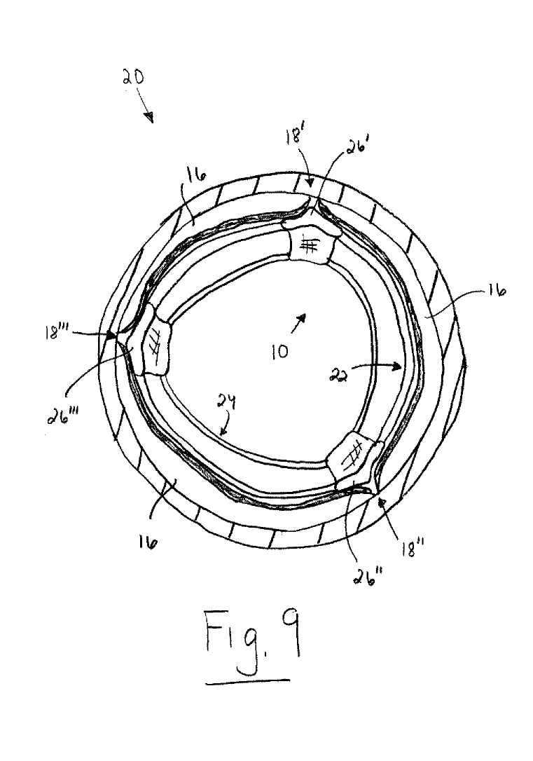

One aspect of the present disclosure includes a device 10 (FIG. 1) for preventing or mitigating paravalvular leakage associated with a valve replacement procedure. As shown in FIG. 1, the device 10 can include a first ring 22, a second ring 24 that is axially spaced apart from the first ring, at least one pad section 26 extending between the first and second rings, and a plurality of centering wires 28 attached to at least one of the first and second rings. The device 10 can be comprised of one or a combination of flexible materials that allow(s) the device to easily transition between a collapsed configuration (FIG. 8) and an expanded configuration (FIG. 1). In the expanded configuration, the device 10 is shaped and dimensioned to snugly fit within a diseased cardiac valve, such as a diseased aortic valve 20.

In another aspect, the first and second rings 22 and 24 are axially spaced apart from one another by a distance D. The distance D can be varied so that each of the first and second rings 22 and 24 can expand into contact with, and apply pressure to, a native cardiac valve annulus (e.g., when the device 10 is in the expanded configuration). The distance D can be varied as needed by the skilled artisan (e.g., based on the particular cardiac valve anatomy of a given patient). In some instances, the first and second rings 22 and 24 can be axially spaced apart so that the distance D is the same or equal between corresponding points on the first and second rings. In such instances, the first and second rings 22 and 24 can be parallel to one another. In other instances, the distance D between a first point on the first ring 22 and a first corresponding point on the second ring 24 may be different than the distance D between a second different point on the first ring a second different corresponding point on the second ring. In such instances, the first and second rings 22 and 24 can be axially offset from one another. In further instances, the first and second rings 22 and 24 can be coaxial with, or substantially coaxial with, one another.

Each of the first and second rings 22 and 24 can have a circular shape as shown in FIG. 1; however, it will be appreciated that the first and second rings can have any shape that allows the rings to expand into contact with, and apply pressure to, a native cardiac valve annulus (e.g., when the device 10 is in the expanded configuration). In some instances, the first ring 22 can be identically shaped as the second ring 24. In other instances, the first ring 22 can have a different shape than the second ring 24. Each of the first and second rings 22 and 24 has a diameter. In some instances, the diameter of the first ring 22 can be equal to the diameter of the second ring 24. In other instances, the diameter of the first ring 22 can be different than the diameter of the second ring 24. The first ring 22 and/or the second ring 24 can comprise a single continuous filament (e.g., a wire). Alternatively, the first ring 22 and/or the second ring 24 can have a braided configuration where, for example, two or more filaments are used to form the ring(s). Each of the first and second rings 22 and 24 can be made of one or a combination of materials that allows the rings to expand into contact with, and apply pressure to, a native cardiac valve annulus. Examples of materials from which the first and second rings 22 and 24 can be made include metals or metal alloys (e.g., stainless steel, Nitinol, titanium, etc.) and polymers (e.g., PTFE).

In another aspect, the device 10 includes at least one pad section 26 that is circumferentially spaced about, and extends longitudinally between, the first and second rings 22 and 24. The number of pad sections 26 included as part of the device 10 can vary depending, for example, on the number of commissures of a diseased heart valve. In one example, the device 10 can include three pad sections 26 where the device is used as part of an aortic valve replacement procedure. The number of pad sections 26, however, need not be dictated by the number of commissures. Thus, the device 10 can include one, two, three, four, or even more pad sections 26. The pad sections 26 can be circumferentially spaced about the first and second rings 22 and 24 by a circumferential distance C_{d}. In one example, the circumferential distance C_{d} between two pad sections 26 can be equal to (or about equal to) the circumferential distance C_{d} between two commissures. In some instances, the circumferential distance C_{d} between each pad section 26 comprising the device 10 can be equal. In other instances, the circumferential distance C_{d} between each pad section 26 comprising the device 10 can be different. In further instances, the circumferential distance C_{d} between first and second pad sections 26, as well as the circumferential distance C_{d} between the first pad section and third pad section, can be the same, whereas the circumferential distance C_{d} between the second and third pad sections can be different.

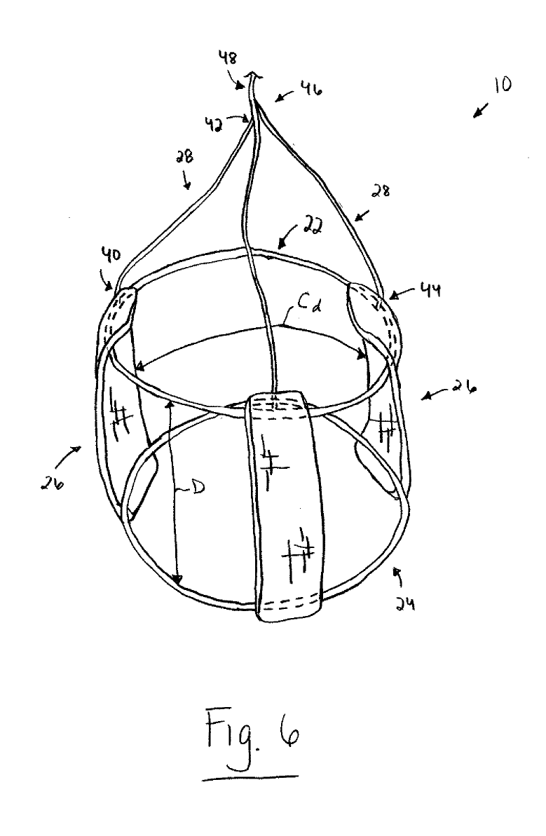

Each pad section 26 (FIG. 4) includes a length L, a width W, and a thickness T, which is defined by oppositely disposed first and second surfaces 30 and 32. As shown in FIG. 4, each pad section 26 can have a rectangular shape (e.g., wherein the length L is greater than the width W). It will be appreciated that each pad section 26 can have other shapes (e.g., square, ovoid, circular, etc.) depending, for example, upon the configuration of the first and second rings 22 and 24. The first surface 30 of each pad section 26 is configured to engage a commissure or commissural region, which can include a commissure per se as well as the tissue surrounding the commissure, such as the leaflets and/or valve annulus. For example, the first surface 30 of each pad section 26 can be configured to contact a commissure or commissural region so that the pad section substantially fills the commissure. Each pad section 26 can be made of one or a combination of materials capable of preventing or mitigating blood leakage through a commissure. In one example, each pad section 26 can be made of one or more materials capable of absorbing blood (e.g., gauze). In another example, each pad section 26 can be made of one or more materials that are semi-permeable or impermeable to blood (e.g., PTFE).

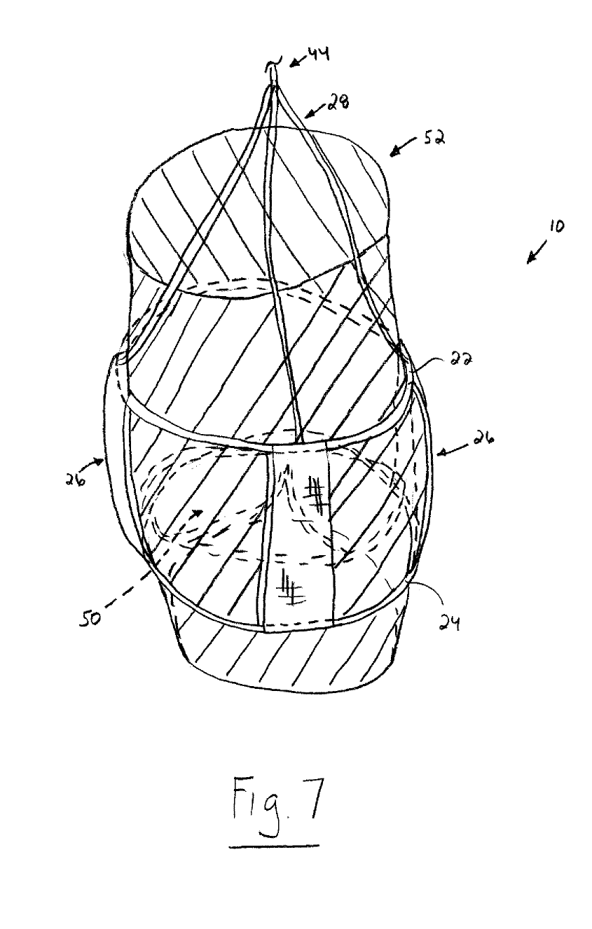

Each pad section 26 can be securely attached to the first and second rings 22 and 24 in one or a variety of configurations, examples of which are illustrated in FIGS. 5A-C. In some instances, an end portion 34 of a pad section 26 can be wrapped around a respective portion of the first ring 22, for example, and be secured thereto using one or more fasteners 36 (e.g., a suture, clip, adhesive, etc.) (FIG. 5A). In other instances, an end 38 of a pad section 26 can be directly attached to a respective portion of the first ring 22, for example, by looping a suture 36 (or other suitable fastener) over the first ring and then threading the suture through the pad section (FIG. 5B). In further instances, the first ring 22 (or the second ring 24) can be threaded directly through an end portion 34 of each pad section 26 (FIG. 5C). In an alternative configuration of the device 10, each pad section 26 (FIG. 6) can extend a distance beyond a radial plane of each of the first and second ring members 22 and 24 to provide optimal coaptation between each pad section and a respective commissure or commissural region.

In another aspect, the device 10 includes a plurality of centering wires 28 attached to the first ring 22 and/or the second ring 24. As described in more detail below, the centering wires 28 can be used assist a medical practitioner (e.g., a surgeon) in positioning and manipulating the device 10 during a valve replacement procedure. In some instances, each of the centering wires 28 can be directly attached to the first ring 22. It will be appreciated, however, that each of the centering wires 28 can be attached to the second ring 24 or, alternatively, that some of the centering wires can be attached to the first ring 22 while other centering wires are attached to the second ring. Although the device 10 in FIG. 1 is shown as having three centering wires 28, it will be appreciated that the device can include any desired number of centering wires. The centering wires 28 can be made of any one or combination of flexible materials, such as stainless steel or Nitinol.

Each of the centering wires 28 includes oppositely disposed first and second ends 40 and 42. In some instances, the first end 40 of each of the centering wires 28 can be directly attached to the first ring 22 at an attachment point 44 near or on the location where a pad section 26 is located. Each centering wire 28 can extend from the first end 40 to the second end 42, which terminates at a central point 46. The second end 42 of each centering wire 28 is attached to the second end of the other centering wires at the central point 46. The second end 42 of each of the centering wires 28 can be attached at the central point 46 in any suitable manner, such as by twisting or soldering the second ends together. From the central point 46, a guide wire 48 extends away from each of the centering wires 28. The guide wire 48 can be used to assist in positioning the device 10 during a valve replacement procedure.

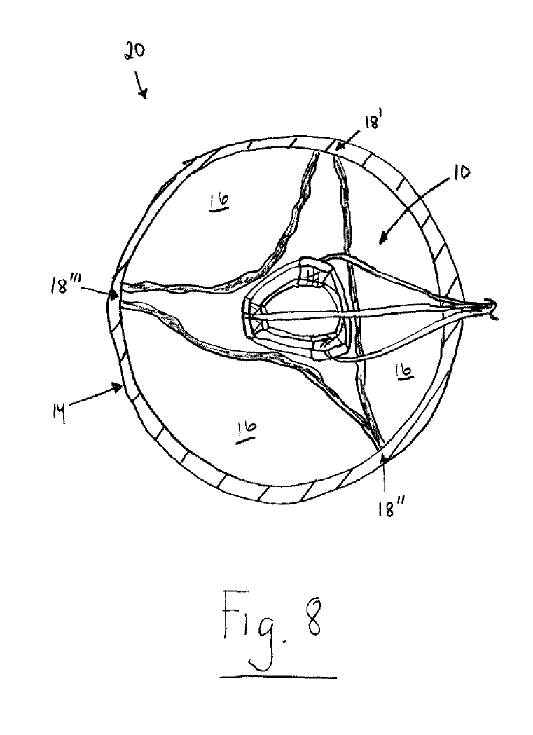

In another aspect, the device 10 can include a prosthetic valve 50 (FIG. 7). In some instances, the prosthetic valve 50 can include a bioprosthetic valve, such as those disclosed in U.S. Patent Pub. Nos. 2006/0195183 A1 and 2006/0259135 A1. In other instances, the prosthetic valve 50 can include a mechanical valve, such as those disclosed in U.S. Pat. Nos. 6,896,700 and 6,875,230. In further instances, the prosthetic valve 50 can include a stented prosthetic valve, such as the one shown in FIG. 7. For example, a stented prosthetic valve can comprise a prosthetic valve component 50 (e.g., a bioprosthetic valve) that is secured within an expandable, stent-like component 52. The prosthetic valve 50 can be secured within the device 10 prior to a valve replacement procedure. Alternatively, the prosthetic valve 50 can be delivered to (and subsequently secured within) the device 10 following implantation of the device.

Another aspect of the present disclosure includes a method for preventing or mitigating paravalvular leakage during a procedure to replace a diseased heart valve. One example of the method is shown in FIGS. 8-9, in which an aortic valve replacement procedure is illustrated. It will be appreciated that the method of the present disclosure is applicable in other valve replacement procedures, such as mitral and tricuspid valve replacement procedures.

One step of the method can include providing a device 10 configured to prevent or mitigate paravalvular leakage. The device 10 can be configured in an identical or similar manner as the device illustrated in FIG. 1. Where the device 10 (FIG. 9) is configured for use in an aortic valve replacement procedure, for example, the device can include a first ring 22, a second ring 24 that is axially spaced apart from the first ring, a first pad section 26′ configured to engage a first commissure 18′ (or commissural region) of a diseased aortic valve 20, a second pad section 26″ configured to engage a second commissure 18″ (or commissural region) of the diseased aortic valve, a third pad section 26″ configured to engage a third commissure 18″′ (or commissural region) of the diseased aortic valve, and three centering wires 28 that are attached to the first ring.

After obtaining an appropriately configured device 10, the device is transitioned into the collapsed configuration (FIG. 8). The device 10 can then be delivered to the diseased aortic heart valve 20 using a minimally invasive, percutaneous, or open heart procedure. For example, the device 10 can be positioned within the diseased aortic valve 20 (as shown in FIG. 8) with the aid of the guidewire 48. Using the centering wires 28, the device 10 can then be rotated (if needed) to align the pad sections 26′, 26″, and 26″ with the commissures 18′, 18″, and 18″ of the diseased aortic valve 20. This can be done by partially expanding the device 10 and then adjusting the lateral and/or vertical position of the device as needed. Depending upon the orientation of the device 10 upon placement in the valve annulus 14, however, there may not be a need to partially expand and/or reposition the device.

Once the device 10 is appropriately positioned within the valve annulus 14, the device can be transitioned into the expanded configuration (FIG. 9). In the expanded configuration, the first surface 30 of each of the pad sections 26′, 26″, and 26″ firmly engages the commissures 18′, 18″, and 18″ (or commissural regions) of the diseased aortic valve 20. Also in the expanded configuration, the first and second rings 22 and 24 can expand into direct physical contact with the valve annulus 14, thereby flattening the valve leaflets 16 against the annulus. As shown in FIG. 9, each of the pad sections 26′, 26″, and 26″ can fill the respective commissures 18′, 18″, and 18″ so that the blood leakage through the commissures is prevented or mitigated. After the device 10 is secured within the aortic valve annulus 14, the centering wires 28 can be removed (e.g., severed) from the device. Although not shown, a prosthetic valve (e.g., a stented prosthetic valve) can then be secured within the device 10 to restore normal blood flow through the valve.

From the above description of the present disclosure, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

1. A device for preventing or mitigating paravalvular leakage associated with a valve replacement procedure, the device comprising:
a first ring;
a second ring that is axially spaced apart from the first ring;
at least one pad section extending between the first and second rings; and
a plurality of centering wires attached to at least one of the first and second rings.

2. The device of claim 1, wherein the first ring is substantially parallel to, and coaxial with, the second ring.

3. The device of claim 1, wherein the first ring and the second ring are each configured to expand into contact with, and apply pressure to, a native valve annulus upon deployment of the device.

4. The device of claim 1, wherein two or more pad sections are circumferentially spaced apart from one another.

5. The device of claim 3, wherein the at least one pad section is disposed about the first and second rings so that the at least one pad section substantially aligns with a commissure of the native valve annulus upon deployment of the device.

6. The device of claim 1, wherein each of the centering wires has a first end that is directly attached to a point on the first ring near a location at which the at least one pad section is located.

7. The device of claim 1, wherein each of the centering wires has a second end that is directly attached to a different centering wire at a central point that is spaced apart from the first and second rings.

8. The device of claim 1, wherein the first and second rings are configured to securely receive a prosthetic valve.

9. The device of claim 1, further including a prosthetic valve secured therein.

10. The device of claim 9, wherein the prosthetic valve is a stented prosthetic valve.

11. A device for preventing or mitigating paravalvular leakage associated with an aortic valve replacement procedure, the device comprising:
a first ring;
a second ring that is axially spaced apart from the first ring;
three pad sections extending between the first and second rings, each of the pad sections having a commissure engaging surface;
a plurality of centering wires attached to at least one of the first and second rings.

12. A method for preventing or mitigating paravalvular leakage during a procedure to replace a diseased heart valve having a valve annulus and at least two commissures, the method comprising the steps of:
providing a device that includes a first ring, a second ring that is axially spaced apart from the first ring, at least one pad section extending between the first and second rings, and a plurality of centering wires attached to at least one of the first and second rings;
deploying the device in the valve annulus so that the at least one pad section engages at least one commissure to form a seal therebetween and thereby reduce or prevent paravalvular leakage; and
deploying a prosthetic valve within the device.

13. The method of claim 12, wherein the step of deploying the device further includes:
positioning the device, in a collapsed configuration, within the valve annulus;
partially expanding the device so that the first and second ring members firmly engage the valve annulus and pin the native valve leaflets against the valve annulus; and
manipulating the centering wires until each of the pad sections is positioned directly adjacent the commissures of the diseased heart valve.

14. The method of claim 12, wherein the prosthetic valve is a stented valve.
